(19) United States
(12) Patent Application Publication
(10) Pub. No.: US 2005/0260145 A1
(43) Pub. Date: Nov. 24, 2005

(54) Oil soluble compositions

(76) Inventors: Steve Leigh, Muttenz (CH); Mathew Louis Steven Leigh, Muttenz (CH); Peter Van Hoogevest, Bubendorf (DE); Henricus Tiemessen, Weil am Rhein (DE)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(21) Appl. No.: 10/516,019

(22) PCT Filed: May 28, 2003
(86) PCT No.: PCT/EP03/05631

§ 371 Date: Jul. 27, 2005

(30) Foreign Application Priority Data
May 30, 2002 (EP) 02253793.0
Oct. 21, 2002 (EP) 02257289.5

(51) Int. Cl. A61K 7/021

(52) U.S. Cl. 424/63

(57) ABSTRACT

A process is disclosed for the preparation of an oily solution of a carotenoid, particularly astaxanthin, a composition comprising at least one xanthophyll and at least one suitable solvent, particularly n-methyl-pyrrolidone, a composition comprising a carotenoid and a mixture of mono-, di- and triglycerides subjected to enzyme treatment, glycerolysis and/or fractionation, and a solid homogeneous mixture comprising one xanthophyll in a physiologically acceptable amount and at least one lipophilic dispersing agent. The process is useful, for example, in preparing nutrient or pharmaceutical compositions.

The present invention relates to a process for the preparation of an oily solution of a pigment additive, a composition comprising at least one xanthophyll and at least one suitable solvent, particularly N-methyl-pyrrolidone, a composition comprising a carotenoid and a mixture of mono-, di- and triglycerides subjected to enzyme treatment, glycerolysis and/or fractionation, and a solid homogeneous mixture comprising one xanthophyll in a physiologically acceptable amount and at least one lipophilic dispersing agent.

Carotenoids are of synthetic or natural origin and are useful as important pigment additives for nutrients and for pharmaceuticals as a substitute for synthetic dyes and antioxidants and may serve as (pro) vitamins.

Carotenoids are classified into two groups: carotenes and xanthophylls. Carotenes, such as beta-carotene or lycopene, are pure hydrocarbons with polyene structure. Xanthophylls additionally contain at least one functional group, such as hydroxy, epoxy or oxo. Representative compounds are astaxanthin, canthaxanthin and zeaxanthin.

Xanthophylls are colorants that are distributed in nature, e.g. in corn (zeaxanthin), green beans (lutein), paprika (capsanthin), egg yolk (lutein) or crustaceans and salmon (astaxanthin).

Carotenoids are almost insoluble in water. Their low solubility in fats and oils and high sensitivity to oxidation impede their use as a colorant in nutrients (food and feed). A sufficient dispersibility is necessary either in the oil or in the water phase to obtain the desired colour effects. Therefore, improved colour effects, when colouring food, are achieved by administering compositions comprising carotenoids in finely dispersed or preferably solubilised form in the optional presence of antioxidants, such as alpha-tocopherol. When used in nutrients these finely dispersed compositions are characterised by their higher bioavailability and improved colouring effects, e.g. when pigmenting egg yolk or fish.

To improve the colour yields and colour effects, numerous attempts have been made to increase the bioavailability of insoluble carotenoids by reducing the particle size to less than 10 μm

Typically, according to U.S. Pat. No. 6,296,877 (BASF) a process for the preparation of a stable aqueous dispersion or water-dispersible dry powder of xanthophylls is disclosed, which comprises
preparing a molecularly dispersed solution of at least one xanthophyll in a water-miscible solvent at a temperature above 30° C.; and
mixing that solution with an aqueous solution of a mixture of suitable hydrophilic protective colloids,
wherein the mixture comprises at least one component of a protective hydrophilic colloid of low molecular weight and at least one component of a protective hydrophilic colloid of higher molecular weight and wherein the molecular weights of the protective colloid components differ by at least 10 000 and forming a nano-dispersed phase; and
preparing a water-dispersible dry powder by removing the solvents in the optional presence of suitable coating materials.

In general, the method is very complicated and yields complex dispersed systems. Extreme temperature conditions need to be used and corresponding equipment able to cope with these extreme conditions is required. Moreover, in order to colour the oil, the dry powder first needs to be dispersed in water and the water phase has to be emulsified with the oil.

An object of the present invention is an improved and simplified method for the preparation of an oily solution comprising higher amounts of xanthophylls resulting in improved colouring effects.

In the description of this invention the following definitions apply:
“Dissolving” means obtaining molecular dispersions and/or colloidal dispersions of the carotenoid. Assessment is by filtration through a 0.45 micrometer pore size filter of such dispersions.
“Lipophilic dispersing agent” is a solid substance with water solubility at room temperature lower than 5 mg/ml which has the property to embed a molecular or colloidal dispersion of the carotenoid in a homogeneous solid composition.
A “homogeneous solid composition” means that the carotenoid is homogeneously distributed in a solid matrix of the lipophilic dispersing agent obtained by simultaneously dissolving the carotenoid and the lipophilic dispersing agent, followed by removal of the solvent.
A “water miscible solvent” means that the solvent can be mixed at any ratio with water without phase separation.
A “water immiscible solvent” means that the solvent can be mixed only partially with an excess of water without phase separation.

The method according to one aspect of the present invention relates to a process for the preparation of an oily solution of a physiologically acceptable pigment additive. The description of the present invention discloses two process variants, namely process variant a) and b). The process comprises process variants
a) preparing a solid composition comprising a carotenoid and dissolving the composition in a physiologically acceptable oil or mixture of oils; or, in the alternative, process variant
b) preparing a solution comprising a carotenoid and an organic solvent; and mixing the solution with a physiologically acceptable oil or mixture of oils followed by the optional removal of the organic solvent.
   Process Variant a)

According to this process variant a solid composition is prepared comprising a carotenoid which may be dissolved in a physiologically acceptable oil or mixture of oils.

The solid composition is prepared by dissolving the carotenoid in an organic solvent with the optional admixture of a lipophilic dispersing agent and further additives; and removing the solvent.

The solid composition obtained in the form of a solid homogeneous mixture comprising
I) At least one carotenoid in a physiologically acceptable amount;
II) At least one lipophilic dispersing agent;
   is also subject matter of the present invention. The solid composition may be used as such in different dosage forms for all types of applications for human, veterinary and aquaculture applications. More preferably, the powder is used to prepare a solution of a carotenoid by dissolving in an oil or oily medium. Furthermore, the oil solution comprising a carotenoid, particularly a xanthophyll, may be used as such in a dosage form for human use internally or externally or incorporated in poultry or fish feed as a colorant.

Suitable carotenoids are astaxanthin, lutein, violaxanthin and neoxanthine cryptoxanthin, canthaxanthin, zeaxanthin, citranaxanthin, beta-carotene, beta-4′-carotenal, beta-8′-carotenal, beta-12′-carotenal, beta-8′-carotenoic acid, lycopene, torularodin aldehyde, torularodin ethyl ester, neurosporaxanthin ethyl ester, zetacarotene, dehydroplectania-xanthin and the like.

The carotenoids, e.g. astaxanthin, are dissolved in a water-immiscible or a water-miscible solvent.

Suitable water-immiscible solvents are, for example, dichloromethane, chloroform, dimethoxymethane, diethoxyethane or dioxacyclopentane.

According to a preferred embodiment of the process the carotenoids are dissolved in a water-miscible solvent.

According to a particularly preferred embodiment of the process the solid composition is prepared in the form of a homogeneous mixture according to process variant a) by dissolving the carotenoid in a water-miscible organic solvent; admixing a lipophilic dispersing agent with optional addition of further additives; and removing the solvent.

Suitable water-miscible solvents are solvents selected from the group consisting of N-methylpyrrolidone (NMP), N,N-dimethylformamide, N,N-dimethylacetamide, 4-formylmorpholine, 4-acetylmorpholine, 4-methylmorpholine and 4-phenylmorpholine or mixtures thereof. Other water-miscible solvents are ethyllactate, glycofurol (tetraglycol), tetrahydrofuran, isopropanol, isopropanol/water (85/15 v/v), ethanol, 96% ethanol, methanol ethyl lactate, polyethylene glycol 300, polyethylene glycol 400, 1,3 butandiol, succinic acid diethyl ester, triethyl citrate, dibutyl sebacate, DMSO, glycerol formal, lactic acid butyl ester, propylene carbonate, propylene glycol diacetate, tetrahydrofurfuryl alcohol, diethylene glycol mono ethyl ether or acetonitrile. Other suitable solvents are 1,1-iminodi-2-propanol and glycerol ketal, e.g. 2-dimethyl-4-oxymethyl-1,3-dioxalan (like Solketal®), lactams, e.g. 2-ketopyrrolidine (Soluphor® P), N-acyl-aminoacid alkyl ester, N-acyl-aminoalcohol acyl ester, acylamino alcohol ester, or amino acid alkyl ester.

Suitable lipophilic dispersing agents are those used as nutrient or as pharmaceutical additives as lipophilic coating material to modify the drug release of oral solid dosage forms.

Suitable dispersing agents may be selected from particular members of the group consisting of ethylcelluloses, synthetic and natural resins, rosins and gums. Preferred are ethylcellulose grades with not less than 44% and not more than 51% by weight of ethoxy groups. More preferred are cellulose grades meeting the requirements of the National Formulary of 48.0-49.5% ethoxy group content (N-grade) (Hercules, Product data brochure on AQUALON® Ethylcellulose). In some cases, however, cellulose grades with less than 44% or more than 51% by weight may be used as well. Depending on their molecular weights, the grade of cellulose or other suitable lipophilic dispersing agent has a viscosity range between about 1.0 to 200.0, preferably 5.6 to 105 cps. Depending on the molecular weights, the grade of cellulose or alternative lipophilic dispersing agent may have viscosities up to 11 cps. Most preferable are ethylcellulose grades with low viscosity, such as N7 or N4 or lower, obtainable from Dow Chemical or Hercules Inc. The viscosity values are obtained from a 5% w/w solution comprising 80 parts toluene and 20 parts ethanol.

The resin Dammar gum is characterised by its low viscosity in organic solvents and is a preferred alternative to low viscosity ethylcellulose. Dammar gum can also be used in combination with low viscosity ethylcellulose. Dammar gum is available with different purity grades, which are defined as D, DD and DDD. The following resins can be used either as the lipophilic excipient on its own or in combination with low viscosity N grade ethylcellulose:
Natural resins: Batu run resin, Congo run resin, Elemi resin, Kauri resin, Manila gum, Mastic gum, rosin wood resin, Sandarc resin, shellac resin, white shellac, Vinsol® resin
Rosin and terpene base resins: Abalyn®, Abitol® E, Cellolyn®21 102M, Ester gum, Hercolyn® D, Lewisol® 28, Pentalyn® A, H, 830. 856, Pentrex® 28, Poly paleresin®, Stabelite® 3, 10 ester Vinsol® ester gum, Zinar®, Zirex® and Zitro®, Uni-Rez® 7200;

The lipophilic dispersing agents mentioned above may be admixed with further additives, such as membrane lipids, or surfactants, such as cationic, anionic or non-ionic surfactants and antioxidants.

In order to protect the carotenoid against oxidation, it is preferred to add as optional components in addition to the lipophilic dispersant agent, antioxidants, membrane lipids, and surfactants. A suitable antioxidant may be selected from the group which consists of alpha-tocopherol, alpha-tocopherol palmitate, alpha-tocopherol acetate, t-butylated hydroxytoluene, t-butylated hydroxyanisole, ascorbic acid and ethoxyquin.

Suitable membrane lipids are phospholipids, which are the most abundant membrane lipids occurring in nature. They can be uncharged, zwitterionic, negatively or positively charged. Examples of uncharged phospholipids are phosphatidyl choline, phosphatidyl ethanolamine or their mono acyl derivatives, sphingomyelin and cholesterol. Examples of negatively charged membrane lipids are phosphatidyl serine, phosphatidyl glycerol, phosphatidic acid, phosphatidyl inositol, cerebrocides, glycolipids cardiolipin. The membrane lipids may be derived from natural plant or animal or microbiological sources, synthesised or partially synthesised, including polyethylene glycol (PEG) derived diacyl and monoacyl equivalents. Special fractions of soy lecithins which contain several phosholipid types, their monoacyl-derivatives, non polar lipids and free fatty acids have improved solubilisation potential and low viscosity due to their heterogeneous composition and may be preferred in some cases. Such blends are commercially available, for example, from Lipoid KG. The term membrane lipid of natural origin also comprises membrane lipids that have been modified by enzymatic action using phospholipase A1, A2 or D.

Surfactants include cationic, anionic or non-ionic surfactants.

A suitable cationic surfactant, is N-benzyl-N,N-dimethyl-N-2-[2-(4-(1,1,3,3-tetramethylbutyl)-phenoxy-ethoxy]-ethylammonio chloride, N-benzyl-N,N-dimethyl-N-2-[2-(3-methyl-4-(1,1,3,3-tetramethylbutyl)-phenoxy)-ethoxy]-ethylammonio chloride (methylbenzethonium chloride), n-dodecyltrimethylammonio chloride or bromide, trimethyl-n-tetradecylammonio chloride or bromide, n-hexadecyltrimethylammonio chloride or bromide (cetyltrimethylammonium chloride or bromide), trimethyl-n-octadecylammonio chloride or bromide, ethyl-n-dodecyidimethylammonio chloride or bromide, ethyldimethyl-n-tetradecylammonio chloride or bromide, ethyl-n-hexadecyltrimethylammonio chloride or bromide, ethyldimethyl-n-octadecylammonio chloride or bromide or n-C12-C16alkyl-benzyldimethylammonio chloride or bromide (benzalkonium chloride or bromide).

Suitable anionic surfactants are sodium or potassium C12-C20-alkylsulphate, e.g. sodium or potassium-n-dodecyl, -tetradecyl, -hexadecyl or octadecylsulphate or -sulphonate, or sodium or potassium C12-C20-alkylethersulphate, e.g. sodium or potassium-n-dodecyloxyethyl, tetradecyloxyethyl, hexadecyloxyethyl or octadecyloxysulphate or -sulphonate.

Suitable non-ionic surfactants are selected from the group consisting of polyglycerol esters, polysorbates, mono- and diglycerides of fatty acids, propylene glycol esters, sucrose fatty acid esters and polyoxyethylene derivatives of sorbitan fatty acid esters. These surfactants are well known in the art and are commercially available.

Another group of suitable surfactant, with HLB values provided in brackets, includes decaglycerol monolaurate [15.5]; decaglycerol distearate [10.5]; decaglycerol dioleate [10.5]; decaglycerol dipalmitate [11.0]; decaglycerol monostearate [13.0]; decaglycerol monooleate [13.5]; hexaglycerol monostearate [12.0]; hexaglycerol monooleate [10.5]; hexaglycerol mono-shortening [12.0]; polyoxyethylene (20) sorbitan monolaurate [16.7]; polyoxyethylene (4) sorbitan monolaurate [13.3]; polyoxyethylene (20) sorbitan monopalmitate [15.6]; polyoxyethylene (20) sorbitan monostearate [14.9]; polyoxyethylene (20) sorbitan tristearate [10.5]; polyoxyethylene (20) sorbitan monooleate [15.0]; polyoxyethylene (5) sorbitan monooleate [10.0]; polyoxyethylene (20) sorbitan trioleate [11.0]. It will be appreciated by those skilled in the art that the HLB value for a surfactant is an expression of its hydrophilic-lipophilic balance, i.e., the balance of the size and strength of the hydrophilic (polar) and lipophilic (non-polar) groups of the surfactant.

Other suitable surfactants which are soluble in NMP are the sucrose esters like, sucrose tristearate, sucrose distearate, sucrose stearate, sucrose palmitate.

Lactic acid derivatives include sodium stearoyl lactylate and calcium stearoyl lactylate.

A particularly preferred surfactant is d-alpha tocopheryl polyethylene glycol-1000-succinate (TPGS).

Other surfactants or substances with amphipathic characteristics may be used as long as they are soluble or miscible in organic solvent. The surfactants suitable for the invention are not limited to the typical examples provided above.

According to a preferred embodiment, low molecular weight surfactants are used, particularly ascorbyl palmitate, polycerol fatty acid esters, sorbitan fatty acid esters, propylene glycol fatty acid esters or membrane lipids and TGPS in a concentration from about 0.0% to 500% by weight, based on the carotenoids.

According to the process of the present invention, administrable compositions comprising carotenoids, particularly xanthophylls, e.g. astaxanthin, are obtained. The process described for e.g. xanthophylls, comprises dissolving the carotenoid in the water-miscible organic solvent, preferably together with at least one lipophilic dispersing agent, e.g. ethylcellulose and/or Dammar gum, at room temperature, or preferably not higher than 90° C. Optionally, at least one antioxidant and/or a low molecular weight amphipathic surfactant is added. The xanthophyll solution is diluted with an excess of water in the absence of protective hydrophilic colloids but optional presence of a low molecular weight amphipathic surfactant.

A precipitate is formed which is dried and, optionally, milled to give a stable solid xanthophyll or a carotenoid composition in the form of a powder, which has an unexpectedly high solubility and dissolution rate in oils. The dry composition may be dissolved directly in most oils at concentrations of at least 250 ppm.

A particularly preferred embodiment of process variant according to a) comprises preparing the solid composition according to a) by dissolving the carotenoid in an organic solvent; admixing a lipophilic dispersing agent selected from the group consisting of ethyl cellulose and lipophilic gums with optional addition of further additives selected from the group consisting of antioxidants and surfactants; and removing the solvent.

The ratio of the lipophilic dispersing agent to xanthophyll is generally selected in such a manner that the solid composition comprises about 0.5 to 20.0% by weight, preferably 5.0 to 15.0% by weight of the carotenoid, from about 10.0 to 99.0%, preferably 20.0 to 85% of lipophilic dispersing agent and from about 0 to 40% by weight of other additives.

To increase the stability of the carotenoid against oxidation, it is advantageous to add stabilizers, such as alpha-tocopherol, tert-butylated hydroxytoluene or tert-butylated hydroxyanisole, ascorbic acid or ethoxyquin. They can be added either to the oil phase or the phase comprising the organic solvent or water phase depending on the solubility of the particular antioxidant in these phases. Preferably they are dissolved together with the carotenoid in the presence of additional additives, such as antioxidants, e.g. alpha tocopherol. The water-miscible organic solvent is removed, for example, by washing the precipitate formed with water or, depending on the boiling point, by evaporation under reduced pressure. The solvent can also be removed at the same time as the water by spray-drying or spray-granulation. Water immiscible solvents are removed by spray drying or spray granulation or similar methods. The dry powder composition obtained is readily soluble in oils.

Alternatively, the carotenoid, lipophilic dispersing agent and optionally an antioxidant are dissolved in a water immiscible solvent followed by removal of the solvent by spray drying or a similar process thereby yielding a solid composition with similar properties to those obtained if a water miscible solvent had been employed.

For the sake of clarity it is to be understood that the lipophilic dispersing agent/s is not used as a coating material in the solid composition in contrast to existing methods. The dry powder comprises a homogeneous mixture of the carotenoid and the lipophilic dispersing agent.

A preferred embodiment of the invention relates to a solid homogeneous mixture comprising
I) At least one xanthophyll selected from the group consisting of astaxanthin, zeaxanthin, cantaxanthin and lutein or mixtures thereof or at least one carotenoid selected from the group consisting of betacarotene and lycopene in physiologically acceptable amounts; and
II) A lipophilic dispersing agent selected from the group consisting of ethyl cellulose, Dammar gum, Batu run resin, Congo run resin, Elemi resin, Kauri resin, Manila gum, Mastic gum, Rosin wood resin, Sandarc resin, shellac resin and white shellac and, optionally further additives selected from the group consisting of antioxidants and surfactants.

The definitions of the components present in the composition and the additional additives correspond to the definitions given above with regard to these components.

The oil in which the carotenoid dry composition is dissolved may be any natural or synthetic, non-toxic, physiologically acceptable oil, or oils such as pure mono-, di- or triglycerides or mixtures thereof. In a preferred embodiment, the hydrocarbon chain is unsaturated or saturated with 12 to 24 carbon atoms. Examples are fish oil, soy oil, or the corresponding mono and diglyceride fractions thereof obtained by complete or partial glycerolysis. Optionally, the oil contains up to 10% by weight of at least one membrane lipid.

Examples of edible oils are specialty marine oils like Norwegian Cod Liver oil (3000A/100D, 2500A/250D) with omega 3-acids-30% (18% EPA/12% DHA), 500% (30% EPA/20% DHA)-, cholesterol-free, high-potency DHA, fish oil of tuna or bonito, fish liver oil, halibut liver oil, pollack liver oil, shark liver oil, squalene, squalane, salmon oil, skipjack liver oil, or edible oils, such as cottonseed oil, sesame oil, coconut oil or peanut oil, almond oil, corn oil, canola oil (rapeseed oil), olive oil, peanut oil (groundnut oil), sunflower oil, safflower oil, vegetable soybean oil, almond oil, apricot kernel, avocado oil, jojoba oil, palm kernel oil, pumpkin seed oil, Castor oil, coconut oil (76 and 110 degrees), sesame oil, toasted sesame oil, flax seed oil (organic, conventional and high lignan), GLA oils (Borage, Black Currant, Evening Primrose), grapeseed oil, hazelnut oil, kukui nut oil, macademia nut oil, mamaku oil, pecan oil, perilla oil, pistachio oil, rice bran oil, tea tree oil, walnut oil, wheat Germ oil, corn oil or canola oil (rapeseed oil).

To illustrate the applicability of the compositions prepared by the process of the invention, a further embodiment relates to compositions for topical application using xanthophylls, such as astaxanthin as antioxidant to protect against UV induced damage from sun exposure. Oils with emollient and lubricious properties may be used to dissolve the carotenoid. The oil may be from natural or synthetic source e.g. fatty acid esters or ethers, such as isopropyl myristate, isopropyl palmitate, myristyl myristic and diisopropyl adipate and medium chain triglycerides.

A particularly preferred embodiment of the process according to a) comprises preparing the solid composition according to a) by dissolving the carotenoid in an organic solvent; admixing a lipophilic dispersing agent selected from the group consisting of ethyl cellulose, Dammar gum, Batu run resin, Congo run resin, Elemi resin, Kauri resin, Manila gum, Mastic gum, Rosin wood resin, Sandarc resin, shellac resin, white shellac, Vinsol® resin, Abalyn®, Abitol® E, Cellolyn®21 102M, ester gum, Hercolyn® D, Lewisol®28, Pentalyn® A, H, 830. 856, Pentrex® 28, Poly paleresin®, Stabelite® 3, 10 ester Vinsol® ester gum, Zinar®, Zirex, Zitro® or Uni-Rez® 7200;

Optionally additional further additives may be selected from antioxidants selected from the group which consists of alpha-tocopherol, alpha-tocopherol palmitate, alpha-tocopherol acetate, t-butylated hydroxytoluene, t-butylated hydroxyanisole, or ethoxyquin and surfactants from separate groups which have e.g. membrane lipids, sucrose esters and TPGS as the representative members; and removing the solvent.

The ratio of the lipophilic dispersing agent to xanthophyll is generally selected in such a manner that the solid composition comprises about 0.5 to 20.0% by weight, preferably 5.0 to 15.0% by weight of the carotenoid, from about 10.0 to 99.0%, preferably 20.0 to 85% of lipophilic dispersing agent and from about 0.0 to 40% by weight of other additives.

To increase the stability of the carotenoid against oxidation, it is advantageous to add stabilizers, such as alpha-tocopherol, tert-butylated hydroxytoluene or tert-butylated hydroxyanisole, ascorbic acid or ethoxyquin. They can be added either to the aqueous phase or the phase comprising the organic solvent. Preferably they are dissolved together with the carotenoid in the optional presence of additional additives, such as emulsifiers, e.g. membrane lipids, cationic, anionic, zwitterionic, uncharged synthetic detergents and TPGS. Depending on the type and amount of the lipophilic dispersing agent and other additives, a deeply coloured liquid is obtained.

Process Variant b)

According to this process variant a solution comprising a carotenoid and an organic solvent is prepared. The solution is mixed with a physiologically acceptable oil or oil mixture followed by the optional removal of the organic solvent.

Suitable solvents are oil-miscible organic solvents, which are preferably water-miscible, such as N-methylpyrrolidone, ethyl acetate or glycofurol. Other suitable solvents are 1,1-iminodi-2-propanol, 1,2-propanediol, ether alcohols, e.g. tetrahydrofurfuryl alcohol-polyethylglycol ether (Glycofurol®) 75), glycerol ketals, e.g. 2-dimethyl-4-oxymethyl-1,3-dioxalane (Solketal®), or lactams, e.g. 2-ketopyrrolidine (Soluphor® P), N-acylamino acid alkyl esters, N-acylamino alcohol acyl esters, acylamino alcohol esters or amino acid alkyl esters. Other water-miscible solvents are, tetrahydrofuran, isopropanol, isopropanol/water (85/15 v/v), ethanol, 96% ethanol, methanol ethyl lactate, polyethylene glycol 300, polyethylene glycol 400, 1,3 butandiol, succinic acid diethyl ester, triethyl citrate, dibutyl sebacate, DMSO, glycerol formal, lactic acid butyl ester, propylene carbonate, propylene glycol diacetate, tetrahydrofurfuryl alcohol, diethylene glycol mono ethyl ether, acetonitrile. Other suitable solvents are 1,1-iminodi-2-propanol and glycerol ketal, e.g. 2-dimethyl-4-oxymethyl-1,3-dioxalan (Solketal®) and lactams, e.g. 2-ketopyrrolidine (Soluphor®) P), N-acyl-amino acid alkyl ester, N-acyl-aminoalcohol acyl ester, acylamino alcohol ester or aminoalkyl ester.

Suitable oil-miscible organic solvents which are not water-miscible are, for example, dichloromethane, chloroform, dimethoxymethane, diethoxyethane or dioxacyclopentane.

Most preferred solvent is N-methylpyrrolidone (NMP) because of its low toxicity, as documented by the U.S. Environmental Protection Agency, and high biodegradability.

Technical qualities of NMP containing 50 ppm methylamine or highly purified NMP can be used.

The oily solution may contain lipophilic dispersing agent/s, such as, e.g. low viscosity grade ethylcellulose, Dammar gum, antioxidant/s, such as alpha tocopherol, membrane lipid/s such as a phospholipid, and surfactant/s, such as cationic, anionic or non-ionic surfactants. According to a preferred embodiment, low molecular weight surfactant/s may be added, e.g. ascorbyl palmitate, polycerol fatty acid esters, sorbitan fatty acid esters, propylene glycol fatty acid-esters or membrane lipids and TGPS.

The weight ratio of the lipophilic dispersing agent to the carotenoid, particularly xanthophyll, is generally from 1:10 to 10:1, preferably 1:1. The concentration of the carotenoid in the solvent should be as high as possible, yielding the highest possible concentration of pigment and lowest possible concentration of solvent in the desired oil. In general, a xanthophyll concentration in the solvent of up to 10% w/v can be achieved, depending on the temperature and conditions employed.

The oil in which the carotenoid solvent mixture is dissolved may be any of the above-mentioned non-toxic, physiologically acceptable oil, or oils such as pure mono-, di- or triglycerides or mixtures thereof. In a preferred embodiment, the hydrocarbon chain is unsaturated or saturated with 12 to 24 carbon atoms. Examples are fish oil, soy oil, or the corresponding mono and diglyceride fractions thereof obtained by complete or partial glycerolysis.

To increase the stability of the carotenoid against oxidation, it is advantageous to add stabilizers, such as alpha-tocopherol, tert-butylated hydroxytoluene or tert-butylated hydroxyanisole, ascorbic acid or ethoxyquin to the oily solution. They can be added either to the oil phase or the phase comprising the organic solvent. Preferably they are dissolved together with the carotenoid in the optional presence of additional additives, such as emulsifiers, e.g. alpha tocopherol.

In a preferred embodiment solvents such as the water and oil-miscible organic solvent described above, are removed, for example, by washing the oil composition with water followed by a phase separation or standing in a storage tank and removing the wash water from the bottom or the oil from the top of the tank. Because of the low log P-octanol/water-value, the preferred organic solvent NMP is easily removed from the oil by washing with an equal volume or less of water. Other separation methods are also applicable e.g. thin film distillation, optionally under reduced pressure. Oil soluble and water-immiscible solvents are preferably removed from the oil phase by thin film distillation and/or vacuum- or convection drying of the final feed composition

The present invention also relates to a composition consisting essentially of
I) At least one carotenoid; and
II) At least one solvent and additional additives selected from the group consisting of lipophilic dispersing agents, lipophilic gums, membrane lipids and surfactants.

The composition is prepared by the method as described above, particularly process variant b). The definitions of the carotenoid, the solvent and the additional additives correspond to the definitions given above with regard to these components.

A preferred embodiment of the invention relates to composition consisting essentially of
I) At least one xanthophyll selected from the group consisting of astaxanthin, zeaxanthin, cantaxanthin and lutein or at least one carotenoid selected from the group consisting of betacarotene and lycopene in physiologically acceptable amounts or mixtures thereof; and
II) At least one water miscible solvent selected from the group consisting of N-methylpyrrolidone (NMP), N,N-dimethylformamide, N,N-dimethylacetamide, 4-formylmorpholine, 4-acetylmorpholine, 4-methylmorpholine and 4-phenylmorpholine.

A particularly preferred embodiment relates to a composition consisting essentially of
I) At least one xanthophyll selected from the group consisting of astaxanthin, zeaxanthin, cantaxanthin and lutein or mixtures thereof in a physiologically acceptable amount; and
II) At least one water miscible solvent selected from the group consisting of N-methylpyrrolidone (NMP), N,N-dimethylformamide, N,N-dimethylacetamide, 4-formylmorpholine, 4-acetylmorpholine, 4-methylmorpholine and 4-phenylmorpholine, ethyl acetate or glycofurol, 1,1-iminodi-2-propanol, 1,2-propanediol, ether alcohols, glycerol ketal, lactam, N-acylamino acid alkyl ester, N-acylamino alcohol acyl esters, acylamino alcohol esters, amino acid alkyl esters, tetrahydrofuran, isopropanol, isopropanol/water, ethanol, methanol, ethyl lactate, polyethylene glycol 300 or 400, 1,3-butanediol, succinic acid diethyl ester, triethyl citrate, dibutyl sebacate, DMSO, acetonitrile, glycerol formal, lactic acid butyl ester, propylene carbonate, propylene glycol diacetate, tetrahydrofurfuryl alcohol, diethylene glycol mono ethyl ether, acetonitrile, 1,1-iminodi-2-propanol and glycerol ketal, lactams N-acylamino acid alkyl ester, N-acylamino alcohol acyl ester, acylamino alcohol ester and amino acid alkyl ester, or mixtures thereof and, optionally, additional additives selected from the group consisting of antioxidants selected from the group consisting of alpha-tocopherol, alpha-tocopherol palmitate, alpha-tocopherol acetate, t-butylated hydroxytoluene, t-butylated hydroxyanisole and ethoxyquin and surfactants selected from the group consisting of membrane lipids, sucrose fatty acid esters and TPGS.

A preferred embodiment of the invention relates to a composition essentially consisting of
I) Astaxanthin optionally admixed with zeaxanthin or canthaxanthin or lutein in physiologically acceptable amounts; and
II) N-Methylpyrrolidone.

The present invention also relates to a process for the preparation of an oily solution of a physiologically acceptable pigment additive, which comprises either
a′) dissolving either a dry powder composition comprising a carotenoid or a carotenoid on its own in an amount effective as a colorant in a mixture of mono-, di- and triglycerides subjected to enzyme treatment, glycerolysis and/or fractionation; or
b′) dissolving a carotenoid in an amount effective as a colorant in a physiologically, acceptable water-miscible solvent and dispersing the solution in a mixture of mono-, di- and triglycerides subjected to enzyme treatment, glycerolysis and/or fractionation.

In this respect a representative example of the glycerolysis reaction is given in JP-B 6-65310.

This reference discloses an alcohol group exchange reaction between a fat and glycerol that is conducted in the presence of an immobilised lipase having 1,3-position selectivity, to obtain diglycerides. According to this embodiment of the invention, the process steps are analogous to the ones mentioned above with regard to the process variants a) and b). The dry powder composition is prepared in a manner similar to the method of process variant a). The oil in which the carotenoid dry composition is dissolved may be any non-toxic, physiologically acceptable oil, such as pure mono-, di- or triglycerides subjected to enzyme treatment, glycerolysis and/or fractionation or mixtures thereof. Triglycerides subjected to any one or more processes involving enzyme treatment, glycerolysis, fractionation, and PUFA (poly unsaturated fatty acids) enrichment may also be used. Such mixtures have improved solvent properties. It has been found that the solubility of xanthophylls, such as astaxanthin is further improved in mixtures of mono, di- and triglycerides resulting from enzyme treatment, e.g. by lipases, glycerolysis, and/or fractionation of long chain fatty acid triglycerides having a chain length between 16 and 26 carbon atoms. Preferably, the fatty acids should be unsaturated or polyunsaturated, containing at least one, two or three double bonds. Most preferably, they should be enriched (more than 20%) with long chain fatty acids of the type found naturally in fish and other forms of marine life, such as poly-unsaturated fatty acids (PUFA's). Natural fish oils contain normally not more than ca 15% of DHA (Docosahexaenoic acid).

The present invention also relates to the composition which is obtained by the method described above. The composition comprises
I) A carotenoid in an amount effective as a colorant; and
II) A mixture of mono-, di- and triglycerides subjected to enzyme treatment, glycerolysis and/or fractionation.

The oily solutions of a physiologically acceptable pigment additive, e.g. carotenoids, e.g. xanthophylls, are eminently suitable as colorants or nutrient additives (food and feed). Typical applications in the feed industry include the administration of astaxanthin pigments in the aquaculture or colouring of egg yolk and broiler skin in poultry farming. The preparations can also be used as food or pharmaceutical additives for specific uses in man.

Another embodiment of the invention relates to oily composition prepared according to process variant a) or b) for human, veterinary and aquaculture applications. The oil solutions which may be used as such as nutriceutical and vitamin supplement or filled into unit dosage forms such as capsules for oral administration.

Yet another embodiment of the invention is a solid or semi-solid composition comprising the oil composition prepared according to process variant a) or b).

The oil compositions can be converted into solid compositions by absorbing the oil solution on excipients from the group comprising starch, modified starch, acacia and tragacanth gum and porous particulate support material selected from the group consisting of, for example, amorphous aluminometasilicate, calcium silicate, silica, including porous silicon dioxide and silicic acid, dibasic anhydrous calcium phosphate and maltodextrin and polystyrene beads/micro-sponges. The resulting solid compositions comprise an oil solution of carotenoid absorbed in a powder matrix. They are particularly suitable as instantaneous water-dispersible powders. Furthermore they may be dispersed in a hydrophilic cream base or a lipophilic ointment base for topical application.

The invention and its advantages will become even more apparent from the following examples. The examples serve for illustrative purposes only and do not limit the scope of the invention.

### EXAMPLES

### Example 1

50 mg of synthetic astaxanthin (96% pure) (Sigma) and 500 mg of Aqualon® ethylcellulose N-10 (Hercules) or Ethocel® Standard 10 premium (Dow) are dissolved in 5 ml N-methyl-pyrrolidone (Fluka). The NMP solution is added under stirring at room temperature to 70 ml water. A precipitate is formed and filtered off by using a G4 glass filter and rinsed with 30 ml water. 19.2 mg of the residue is dried in a vacuum at 30° C. The dry powder may be used as such on its own as dosage form e.g. for oral administration and is suitable for long term storage and transport. More usually in order to achieve a 250 ppm solution of the xanthophyll, 19.2 mg of the resulting powder is dissolved at room temperature under stirring in 7 ml fish oil (Egersund Sildoljefabrikk) containing 2.5% Soy Lipid S 20 (Lipoid). After filtration through a 0.45μ Rotilabo®—(PVDF) filter to remove only un-dissolved particles, spectrophotometric analysis at the absorption maximum of astaxanthin at 480 nm of the oily solution, showed that the astaxanthin is dissolved in the oil. Optionally, the oil solution containing dissolved xanthophyll is added to feed, e.g. sprayed as a solution, into fish feed, or in the alternative, to feed or food or admixed with pharmaceutical excipients for oral or topical use. Furthermore, the compositions may be contained in a capsule or a unit dosage form for oral use as a food supplement or nutrient.

### Example 2

In a manner analogous to Example 150 mg of synthetic astaxanthin (Sigma), 500 mg Aqualon® ethylcellulose N-10 (Hercules) and 250 mg TGPS (d-α-tocopherol polyethylene glycol-1000-succinate, Eastman) are dissolved in 5 ml N-methyl-pyrrolidone (Fluka) at room temperature. A dry powder is obtained by adding the NMP solution to water, washing and filtering the precipitate formed. In order to obtain a 250 ppm xanthophyll concentration, 25.2 mg of the resulting powder is dissolved under stirring at room temperature in 7 ml fish oil (Egersund Sildoljefabrikk) containing 2.5% soy lecithin (S 20) (Lipoid)

### Example 3

In a manner analogous to Example 1 50 mg synthetic astaxanthin (Sigma), 500 mg Aqualon® ethylcellulose N-10 (Hercules), 50 mg alpha-tocopherol (Fluka) and 50 mg soy phospholipid S-20 (Lipoid) are dissolved at room temperature in 5 ml N-methyl-pyrrolidone (Fluka). A dry powder is obtained by adding the NMP solution to water, washing and filtering the precipitate formed. In order to obtain a 250 ppm xanthophyll concentration, 19.4 mg of the resulting powder is dissolved at room temperature under stirring in 7 ml fish oil (Egersund Sildoljefabrikk).

### Example 4

In a manner analogous to Example 1, oil soluble solid compositions 1-7 are prepared. The Compositions are specified in the following Table (weight parts). The dilution factor of the NMP phase with water is mentioned, too.

| Composition | 1 | 2 | 3 | 4 | 5 | 6 | 7 |
|---|---|---|---|---|---|---|---|
| Astaxanthin (Sigma) | 1 | 1 | 1 | 1 | 1 | 1 | 1 |
| Alpha-tocopherol (Fluka) | 1 | 1 | 1 | 1 | 1 | 1 | 1 |
| AQUALON Ethylcellulose | 10 | | | | | | |
| N10 (Hercules) or ETHOCEL | | | | | | | |
| Ethylcellulose Standard 10 | | | | | | | |
| Premium (Dow) | | | | | | | |
| AQUALON Ethylcellulose N7 | | 10 | | | | | |
| (Hercules) or ETHOCEL | | | | | | | |
| Ethylcellulose Standard 7 | | | | | | | |
| Premium, (Dow) | | | | | | | |
| AQUALON Ethylcellulose N4 | | | 10 | 10 | 10 | 10 | 8 |
| (Hercules) or ETHOCEL | | | | | | | |
| Ethylcellulose Standard 4 | | | | | | | |
| Premium, (Dow) | | | | | | | |
| Water/NMP ratio | 14 | 14 | 14 | 10 | 7 | 5 | 3 |

### Example 5

Lycopene (1 weight part) (Sigma) is dissolved in NMP at 2.5 mg/ml at room temperature. 1 weight part of alpha tocopherol (Roche) and 10 weight parts of Ethylcellulose ETHOCEL N4 (Dow) are added. The resulting solution is injected into 20 fold excess by volume of water under stirring (ultra-turrax). The precipitate is filtered off and washed with water to remove the residual NMP and dried in an oven under vacuum to remove the water. The resulting solid composition is soluble in soy oil.

### Example 6

100 mg astaxanthin (Sigma), 100 mg alpha tocopherol and 1000 mg Aqualon® ethylcellulose N-10 (Hercules) are dissolved in 20 ml dichloromethane (Riedel de Haen) and sprayed onto a glass surface from an air driven paint sprayer. The resulting dry films are dried further overnight at RT under vacuum. The dry product is collected from the glass surface. The dry powder is suitable for long term storage and transport. To obtain 250 ppm xanthophyll solution, 19.2 mg of the dry product is dissolved at room temperature under stirring in 7 ml fish oil (Egersund Sildoljefabrikk) containing 2.5% soy lipid S 20 (Lipoid). Spectrophotometric analysis at 480 nm of the oil solution showed that the Astaxanthin is dissolved. Optionally, the oily phase containing the dissolved xanthophyll is sprayed or added to fish feed.

### Example 7

In a manner analogous to Example 6, the oil soluble solid compositions 1-5 are prepared. The table specifies the weight parts of the components.

| Composition | 1 | 2 | 3 | 4 | 5 |
|---|---|---|---|---|---|
| Astaxanthin (Sigma) | 8.3 | 7.7 | 14.9 | 7.7 | 1.0 |
| Ethylcellulose N10 (Hercules) | 83.3 | 77.4 | 74.7 | 61.5 | |
| Lecithin S20 (Lipoid) | | | | | 99.0 |
| Alpha-Tocopherol (Fluka) | 8.3 | 7.4 | | | |
| Ethoxyquin (Fluka) | | | 4.6 | | |
| BHT (Fluka) | | 7.4 | | | |
| TPGS (Vit E-PEG) d-α-Tocopherol | | | | 30.8 | |
| Polyethylene Glycol 1000 Succinate | | | | | |
| (Eastman) | | | | | |

### Example 8

In a manner analogous to Example 6, the oil soluble solid compositions 1-5 are prepared. Instead of using a paint sprayer, a spray dryer is used to manufacture 1.5 kg batches. The table specifies the weight parts of the components.

| Composition | 1 | 2 | 3 | 4 | 8 |
|---|---|---|---|---|---|
| Astaxanthin (Sigma) | 1 | 1 | 1 | 1 | 1 |
| Alpha-Tocopherol (Roche) | 1 | 1 | 1 | 1 | 1 |
| Ethylcellulose N10 (Hercules) or | 10 | 5 | | | |
| ETHOCEL Standard 10 Premium (Dow) | | | | | |
| Ethylcellutose N7 (Hercules) or | | | 10 | | |
| ETHOCEL Standard 7 Premium (Dow) | | | | | |
| Ethylcellulose N4 (Hercules) or | | | | | 8 |
| ETHOCEL 4 Premium (Dow) | | | | | |
| Dammar Gum (Resinogum ® DD IRX) | | | | 10 | |
| (CNI) | | | | | |

### Example 9

Beta-carotene (Sigma) (1 weight part) is dissolved in methylene chloride (Riedel de Haen) at a concentration 6.7 mg/ml. 1 weight part of alpha tocopherol and 8 weight parts of ETHOCEL Ethylcellulose N4 (Dow) are added and dissolved and the methylene chloride solution sprayed onto a glass surface from the pressurised sprayer. The resulting dry films are dried further overnight at room temperature under vacuum. The dry product is collected from the glass surface. The dry powder is suitable for long term storage and transport and soluble in oil.

### Example 10

The oil solution containing astaxanthin is prepared as follows: 1.0 g astaxanthin and 1.0 g alpha tocopherol (Roche) are dissolved at 60° C. temperature in 98 ml N-methyl-pyrrolidone (BASF). The NMP solution is mixed with an excess of fish oil (Egersund Sildoljefabrikk). The NMP solution is pumped at a rate of 250 ml (i.e. 2.5 g Astaxanthin)/min and the oil at 10 l/min into an in line rotor stator mixer. The oil containing 250 ppm xanthophyll is sprayed on to a fish feed composition using a 3 to 7 weight ratio (oil/feed).

### Example 11

The oil dispersion containing astaxanthin is prepared as follows: 5 parts by weight astaxanthin (Sigma) and 5 part by weight alpha tocopherol (Roche) are suspended in 90.0 part by weight NMP and vigorously mixed in a closed vessel at about 90° C. for a short time period. The pigment is partly in solution and partly in colloidal suspension and processed further with an excess of fish oil. The oil composition containing 250 ppm xanthophyll is sprayed onto a fish feed composition using a 3 to 7 weight ratio (oil/feed).

### Example 12

In a manner analogous to Examples 10 or 11 a mono, di or tri glyceride derivative of fish or vegetable triglycerides, edible oils or oils suitable for topical administration may be used in place of natural fish oil as replacement: in any of the preceding examples. Furthermore, the compositions may be contained in a capsule or a unit dosage form for oral use as a food supplement or nutrient or included in a topical dosage form.

### Example 13

One part by weight astaxanthin (Sigma) is dissolved in 2500 parts by weight of a vegetable oil previously subjected to glycerolysis, containing a mixture of mono, diglycerides and triglycerides and 1000 parts by weight of a phosphatidyl choline enriched phospholipid. The resultant deep reddish solution is clear and generates an orange dispersion upon dilution with water. The oily concentrate is added to fish feed to obtain a concentration of 60 ppm pigment in the feed. Furthermore, the compositions may be contained in a capsule or a unit dosage form for oral use as a food supplement or nutrient or included in a topical dosage form.

### Example 14

One part by weight astaxanthin (Sigma) is dissolved in 3000 parts by weight natural fish oil containing triglycerides enriched with 70% DHA (Docosahexaenoic acid) (Algatrium, Spain). Upon dilution with water an orange dispersion is obtained. The oily concentrate may be used as a pigment additive in food and feed production to give the required colour. Furthermore, the compositions may be contained in a capsule or a unit dosage form for oral use as a food supplement or nutrient or included in topical dosage forms.

### Example 15

1 part by weight canthaxanthin is dissolved in 3500 parts by weight natural fish oil, containing a mixture of mono, diglycerides and triglycerides. In this solution, 500 parts by weight of a deoiled enzyme modified hydrolysed PC phospholipid (Lipoid) is dispersed. The resultant oily liquid is hydrated readily when added to water and produces an orange coloured dispersion. The oily concentrate is used in fish feed production to give a pigment concentration 40 ppm. Furthermore, the compositions may be contained in a capsule or a unit dosage form for oral use as a food supplement or nutrient or included in a topical dosage forms

### Example 16

In place of astaxanthin any other xanthophyll or mixtures thereof, e.g. lutein, canthaxanthin, zeaxanthin, violaxanthin, neoxanthine, caryptoxanthin, citranaxanthin, may be used as replacement in any of the preceding examples.

1. A process for the preparation of an oily solution of a physiologically acceptable pigment additive, which comprises:
a) preparing a solid composition comprising a carotenoid; and dissolving the composition in a physiologically acceptable oil or oily mixture; or
b) preparing a solution comprising a carotenoid and an organic solvent; and mixing the solution with a physiologically acceptable oil or oil mixture followed by the optional removal of the organic solvent.

2. A process according to claim 1, which comprises preparing the solid composition according to a) by dissolving the carotenoid in a water-miscible organic solvent; admixing a lipophilic dispersing agent with optional addition of further additives; and removing the solvent.

3. A process according to claim 1, which comprises preparing the solid composition according to a) by dissolving the carotenoid in an organic solvent; admixing a lipophilic dispersing agent selected from the group consisting of ethyl cellulose, Dammar gum, Batu run resin, Congo run resin, Elemi resin, Kauri resin, Manila gum, Mastic gum, Rosin wood resin, Sandarc resin, shellac resin and white shellac with optional addition of further additives selected from the group consisting of antioxidants from the group which consists of alpha-tocopherol, alpha-tocopherol palmitate, alpha-tocopherol acetate, t-butylated hydroxytoluene, t-butylated hydroxyanisole, or ethoxyquin and surfactants from the groups which consist of membrane lipids and sucrose fatty acid esters and TPGS; and removing the solvent.

4. A process according to claim 1, which comprises preparing a solution of the carotenoid according to b) in a water-miscible solvent selected from the group consisting of N-methylpyrrolidone (NMP), N,N-dimethylformamide, N,N-dimethylacetamide, 4-formylmorpholine, 4-acetylmorpholine, 4-methylmorpholine and 4-phenylmorpholine acetonitrile, ethyl acetate or glycofurol, 1,1-iminodi-2-propanol, 1,2-propanediol, ether alcohols, tetrahydrofurfuryl alcohol-polyethylglycol ether, glycerol ketals, lactams, N-acylamino acid alkyl esters, N-acylamino alcohol acyl esters, acylamino alcohol esters or amino acid alkyl esters, tetrahydrofuran, isopropanol, isopropanol/water, ethanol, methanol ethyl lactate, polyethylene glycol 300 or 400,1,3-butanediol, succinic acid diethyl ester, triethyltrate, dibutyl sebacate, DMSO, glycerol formal, lactic acid butyl ester, propylene carbonate, propylene glycol diacetate, tetrahydrofurfuryl alcohol, diethylene glycol mono ethyl ether, acetonitrile, 1,1-iminodi-2-propanol, glycerol ketal, lactams, N-acylamino acid alkyl ester, N-acyl-aminoalcohol acyl ester, acylamino alcohol ester and amino acid alkyl ester or mixtures thereof.

5. A process for preparation of an oily solution of a physiologically acceptable pigment additive, which comprises:
a′) subjecting a mixture of mono-, di- and triglycerides to enzyme treatment, glycradysis and/or fractionation; and dissolving either a dry powder composition comprising a carotenoid or a carotenoid on its own in an amount effective as a colorant in the mixture of mono-, di- and triglycerides subjected to enzyme treatment, glycerolysis and/or fractionation; or
b′) dissolving either a dry powder composition comprising a carotenoid or a carotenoid on its own in an amount effective as a colorant in a physiologically, water-miscible solvent and dispersing the solution in a mixture of mono-, di-; and
c′) triglycerides subjected to enzyme treatment, glycerolysis and/or fractionation.

6. A process according to claim 1, wherein the carotenoid is a xanthophyll selected from the group consisting of astaxanthin, canthaxanthin, lutein and zeaxanthin.

7. A process according to claim 1, wherein the carotenoid is a carotene selected from the group consisting of betacarotene and lycopene.

8. A composition consisting essentially of:
I) At least one carotenoid; and
II) At least one water miscible solvent and additional additives selected from the group consisting of lipophilic dispersing agents, lipophilic gums, membrane lipids and surfactants.

9. A composition according to claim 8 consisting essentially of:
I) At least one xanthophyll selected from the group consisting of astaxanthin, zeaxanthin, cantaxanthin and lutein or at least one carotenoid selected from the group consisting of betacarotene and lycopene in physiologically acceptable amounts or mixtures thereof; and
II) At least one water miscible solvent selected from the group of N-methylpyrrolidone (NMP), N,N-dimethylformamide, N,N-dimethylacetamide, 4-formylmorpholine, 4-acetylmorpholine, 4-methylmorpholine and 4-phenylmorpholine.

10. A composition according to claim 8 consisting essentially of:
I) At least one xanthophyll selected from the group consisting of astaxanthin, zeaxanthin, cantaxanthin and lutein or mixtures thereof in a physiologically acceptable amount; and
II) At least one water miscible solvent selected from the group consisting of N-methylpyrrolidone (NMP), N,N-dimethylformamide, N,N-dimethylacetamide, 4-formylmorpholine, 4-acetylmorpholine, 4-methylmorpholine and 4-phenylmorpholine, ethyl acetate or glycofurol, 1,1-iminodi-2-propanol, 1,2-propanediol, ether alcohols, glycerol ketal, lactam, N-acylamino acid alkyl ester, N-acylamino alcohol acyl esters, acylamino alcohol esters, amino acid alkyl esters, tetrahydrofuran, isopropanol, isopropanol/water, ethanol, methanol, ethyl lactate, polyethylene glycol 300 or 400, 1,3 butanediol, succinic acid diethyl ester, triethyl citrate, dibutyl sebacate, DMSO, acetonitrile, glycerol formal, lactic acid butyl ester, propylene carbonate, propylene glycol diacetate, tetrahydrofurfuryl alcohol, diethylene glycol mono ethyl ether, acetonitrile, 1,1-iminodi-2-propanol and glycerol ketal, lactams N-acylaminoacid alkyl ester, N-acylaminoalkohol acyl ester, acylamino alcohol ester and aminoacid alkyl ester, or mixtures thereof and, optionally, additional additives selected from the group consisting of antioxidants selected from the group consisting of alpha-tocopherol, alpha-tocopherol palmitate, alpha-tocopherol acetate, t-butylated hydroxytoluene, t-butylated hydroxyanisole and ethoxyquin and surfactants selected from the group consisting of membrane lipids, sucrose fatty acid esters and TPGS.

11. A composition according to claim 8 consisting essentially of:
I) Astaxanthin optionally admixed with zeaxanthin, canthaxanthin or lutein in physiologically acceptable amounts; and
II) N-Methylpyrrolidone.

12. A composition comprising:
I) A carotenoid in an amount effective as a colourant; and
II) Triglycerides subjected to any one or more processes involving enzyme treatment, glycerolysis, fractionation, and PUFA enrichment.

13. A solid homogeneous mixture comprising:
I) At least one carotenoid in a physiologically acceptable amount; and
II) At least one lipophilic dispersing agent.

14. A solid homogenous mixture according to claim 13 comprising:
I) At least one xanthophyll selected from the group consisting of astaxanthin, zeaxanthin, cantaxanthin and lutein or mixtures thereof or at least one carotenoid selected from the group consisting of betacarotene and lycopene in physiologically acceptable amounts; and
II) A lipophilic dispersing agent selected from the group consisting of ethyl cellulose, Dammar gum, Batu run resin, Congo run resin, Elemi resin, Kauri resin, Manila gum, Mastic gum, Rosin wood resin, Sandarc resin, shellac resin and white shellac.

15. A solid homogeneous mixture according to claim 14 for at least one of human, veterinary and aquaculture applications.

16. A solid or semi-solid composition comprising an oil composition prepared according to the process of claim 1.

17. An oily composition prepared according to claim 1 for at least one of human, veterinary and aquaculture applications.

18. A process according to claim 5, wherein the carotenoid is a xanthophyll selected from the group consisting of astaxanthin, canthaxanthin, lutein and zeaxanthin.

19. A process according to claim 5, wherein the carotenoid is a carotene selected from the group consisting of betacarotene and lycopene.

20. A solid homogeneous mixture according to claim 14, comprising: additives selected from the group consisting of antioxidants and surfactants.
